(19) United States
(12) Patent Application Publication
(10) Pub. No.: US 2015/0290419 A1
(43) Pub. Date: Oct. 15, 2015

(54) DEVICES, SYSTEMS, AND METHODS FOR AUTOMATED ENHANCED CARE ROOMS

(71) Applicant: Elwha LLC, Bellevue, WA (US)

(72) Inventors: Jordin T. Kare, Seattle, WA (US); Wayne R. Kindsvogel, Seattle, WA (US); Elizabeth A. Sweeney, Seattle, WA (US)

(21) Appl. No.: 14/535,689

(22) Filed: Nov. 7, 2014

Related U.S. Application Data
(63) Continuation of application No. 14/251,984, filed on Apr. 14, 2014.
Application No. 14/251,984 is a continuation-in-part of application No. 14/252,098, filed on Apr. 14, 2014.
Application No. 14/252,098 is a continuation-in-part of application No. 14/252,158, filed on Apr. 14, 2014.

(51) Int. Cl. A61M 21/02

(52) U.S. Cl. CPC A61M 21/02; A61M 2021/0066

(57) ABSTRACT

Disclosed are various embodiments of systems and methods related to determining beneficial and customized surroundings for a subject in a healthcare setting or related type setting, based at least in part on the subject's personal profile including one or more of personal health information, detected biological data, input from the subject itself, or input from a healthcare worker.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority Application(s)).

### PRIORITY APPLICATIONS

None.

If the listings of applications provided above are inconsistent with the listings provided via an ADS, it is the intent of the Applicant to claim priority to each application that appears in the Domestic Benefit/National Stage Information section of the ADS and to each application that appears in the Priority Applications section of this application.

All subject matter of the Priority Applications and of any and all applications related to the Priority Applications by priority claims (directly or indirectly), including any priority claims made and subject matter incorporated by reference therein as of the filing date of the instant application, is incorporated herein by reference to the extent such subject matter is not inconsistent herewith.

### SUMMARY

Various embodiments are disclosed herein that relate to methods, devices, systems, and computer program products for automated alteration of Environmental Supplements as part of a dynamic system of medical care rooms or long term care facility rooms or other living spaces that enhance or promote physical, social, and/or psychological health or well-being of a subject therein. Various embodiments provide suggestions or options for altering the subject's surroundings based on biological data collected from the subject by various means, including one or more sensors. Various embodiments describe generating a profile for the subject as an initial determination as to which Environmental Supplement(s) are expected to enhance the subject's health, the profile based in part on health history, input by the subject itself, or input by others (e.g., health care workers).

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a partial view of a disclosed embodiment.

FIG. 2 is a partial view of a disclosed embodiment.

FIG. 3 is a partial view of a disclosed embodiment.

FIG. 4 is a partial view of a disclosed embodiment.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Various embodiments disclosed herein relate to creating healthier environments related to physical, psychological, or social health. In an embodiment, creating healthier environments reduces readmission rates to the healthcare facility. In an embodiment, an otherwise stressful, unfamiliar, or chaotic environment related to traditional medical or healthcare settings, or long term care facilities are improved for better health, as measured by various means described herein. In an embodiment, the setting includes at least one of a hospital inpatient room, hospital or clinic outpatient room, labor/delivery room, neonate room, intensive care unit, emergency room, psychiatric ward/room, pediatric ward/room, diagnostic ward/room, examination ward/room, cancer treatment center, Alzheimer's unit, eldercare unit, or long term care facility (e.g., nursing home, daycare, hospice, assisted living facility, group home, etc.) with a room, apartment, or other area occupied by a subject.

In an embodiment, the subject includes a human (e.g., child, elder, pregnant woman, ill person, terminally ill person, etc.) or companion animal (e.g., dog, cat, etc.).

Subjects that are hospitalized with traditional settings often experience a post-hospital syndrome, or period of physical, psychological, or social health vulnerability due to the allostatic and physiological stress that is experienced during the stay in the short term or even long term care facilities. In certain instances, the subject's health is so compromised by the foreign setting and unpredictable schedule with often many sleep interruptions and interruptions to irregular daily habits (e.g., eating, sleeping, resting, exercising, etc.) that the subject is re-admitted to the hospital or other healthcare facility with another ailment quickly following release from the healthcare facility. See for example, Krumholz, N. Engl. J. Med. 2013; 368:100-102, which is incorporated herein by reference. Anxiety and agitation are important stress factors for many ill patients. A subject's physical, psychological, and social health declines during this period of stress, leaving the subject vulnerable to infection and other afflictions. In addition, lack of mental and physical stimulation can lead to mental defects, particularly in the elderly. In an embodiment, the avoidance of re-admission as well as the improvement in the overall healthcare, including cognition, of the subject can be enhanced by altering the subject's environment so that the subject is more comfortable, calm, content, and engaged.

In an embodiment, a dynamic and programmable system includes a responsive Environmental Supplement based on a personal profile related to a specific subject. In an embodiment, the personal profile of a subject includes at least one of personal medical history or other medical information, input from the subject itself, detected biological data, or input from at least one healthcare worker. In an embodiment, the personal profile includes input information derived from at least one first sensor. In an embodiment, the input includes information provided through interaction with the subject (e.g., by questionnaire). The questionnaire may include various questions related to the physical, social and/or psychological health of the subject.

In an embodiment, the data related to the subject includes at least one of sensed data, stored data, or broadcast data, depending on the particular sensor(s) employed. In an embodiment, input data includes information regarding a situational awareness (e.g., time, date, location, etc.). In an embodiment, the input data related to the subject includes manually input information as disclosed herein.

In an embodiment, the biological data includes biofeedback (e.g., pain perception or muscle improvements) that is utilized to improve health or physiological changes related to increased mobility or range of motion, etc. based on one or more sensors.

In an embodiment, the biological data includes biometric data (e.g., physiological or behavioral characteristics such as face recognition, gait, voice, etc.).

In an embodiment, the input information is derived from a series or sequence of sensors, including sensor arrays. For example in an embodiment a first round of sensors provides a first round of information related to the physical, psychological or social health of the subject. Based on the outcome of the first round, a second round of sensors is selected and receipt of a second round of information related to the subject is attained. Third, fourth, or fifth rounds of sensing information related to the subject may be conducted in this manner, or a full cycle return to the first round of sensors may be initiated at a given time point or based on the information attained in one or more rounds of sensor activation.

Various sensors are described herein and relate to particular embodiments disclosed. For example, non-physiological or physiological sensors (e.g., breath analyzer, heart rate monitor, blood pressure monitor, body temperature monitor, etc.) may be utilized with the subject in addition to or instead of one or more biological assays in the system (e.g., blood glucose, breath analyzer, DNA swab, etc.), as well as self-reporting of general health or ailment symptoms. In an embodiment, the subject is given a choice as to whether to be assessed by any particular sensor. In addition, the personal profile may include information received from the subject's personal health record (e.g., electronic record).

Finally, the personal profile may also include information input from a healthcare worker (e.g., doctor, nurse, aid, etc.) based on observations of the subject. Based on the various input, the personal profile will be “matched” to the Environmental Supplement database.

In an embodiment, the Environmental Supplement database includes at least one dynamic temporal or spatial image (e.g., traveling on land, sea, or air, between indoors and outdoors, or daylight turning to darkness). In an embodiment, the at least one dynamic temporal or spatial image includes historical or stored data. In an embodiment, the at least one dynamic temporal or spatial image includes real-time data that has been detected by one or more sensors, with or without object tracking. In an embodiment, the at least one dynamic temporal or spatial image includes computer-generated data, for example based on historical, stored, or sensor-detected information, with or without prediction or extrapolation. In an embodiment, the Environmental Supplement database includes at least one dynamic temporal or spatial sound (e.g., birds chirping as sunrise nears, crickets chirping at moonlight, or rain increasing or decreasing over time), temperature (increasing or decreasing over time), or other environmental characteristic.

In an embodiment, the Environmental Supplement database includes neighborhood or personal home components that provide a sense of order or structure to the subject's environment. In an embodiment, the components from the subject's neighborhood or personal home include historical or stored data. In an embodiment, the components from the subject's neighborhood or personal home include real-time data that has been detected by one or more sensors. In an embodiment, the components from the subject's neighborhood or personal home include computer-generated data, for example based on historical, stored, or sensor-detected information, with or without prediction or extrapolation.

In an embodiment, the environment is altered dynamically by real-time sensed information that is based on one or more of external sensors (external environment) or sensors monitoring the subject itself. In an embodiment, the external environment sensors include non-conductive remote sensors with regard to the subject. In an embodiment, an external environment (for example, an indoor or outdoor environment, a public or private environment, or a familiar or unfamiliar environment) is mimicked as informed by one or more sensors (e.g., a camera, a microphone, a weather sensor, a thermometer, or other sensor) that provide real-time guidance to dynamically alter the subject's surroundings (e.g., the ceiling may reflect what the sky above it looks like, in real time as the day progresses or over a chosen time period, to give the sense of being outdoors). In an embodiment, the external environment data is predicted based on historical or stored information. In an embodiment, the external environment data is predicted based on broadcast data (e.g., internet or television broadcast, or closed circuit television), which might include public broadcast data. In an embodiment, the external environment data includes data related to time or date, including cyclic, seasonal, or calendar data. In an embodiment, the subject's home environment is mimicked such that smart home sensors provide real-time guidance to dynamically alter the subject's surroundings with Environmental Supplements such that the subject “feels at home” by way of the subject's home mimicked in real-time. In an embodiment, the subject's home environment data is predicted based on historical or stored information. In an embodiment, a subject's favored environment, such as a favorite vacation environment or past-time environment (e.g., a golf course) is mimicked to provide a sense of enjoyment. In an embodiment, the subject's favored environment is provided using historical or stored information, real time information from sensors, or data broadcast (e.g., internet or television broadcast, or closed circuit television), which might include public broadcast data.

In an embodiment, the one or more sensors are in operable communication with other sensors or other components of the systems described herein. In an embodiment, the one or more sensors are in wireless communication with at least one other sensor or other component of the system. Various modes of wireless communication are disclosed herein. In an embodiment, information attained from one component or aspect of the system is shared or transferred to another part of the system. For example, a blood pressure sensor transmits signals to another component of the system which activates a heart rate monitor or other sensor. Further, a healthcare worker may enter observations of the subject into the system, thus the system represents in an embodiment an integrated system of multi-directional communication between a subject, a healthcare worker, one or more sensors or other input devices, and one or more databases for determining the dynamic Environmental Supplement pre-determined to provide the most beneficial environment for the overall health of the subject.

In an embodiment, the systems, devices, methods, or computer program products described herein include the ability to interact with input or additional information from, for example, another computer system or dataset (e.g., personal data storage), personal monitoring device or sensor network, patient tracking (e.g., Amelior EDTracker), information system (e.g., Amelior ED), network system sensors (e.g., mT Tag™, etc.). See for example, U.S. Pat. App. Pub. Nos. 2007/0088713 and 2006/0212085, each of which is incorporated herein by reference.

In an embodiment, the system (by way of hardware, software, etc.) includes the subject's health history, for example as input information from the subject, as accessed by way of electronic health record, or as input information from a healthcare worker or a person other than the subject.

In an embodiment, the system includes a public or “crowdsourcing” database utilized in whole or in part for comparison with the Environmental Supplement offered to or selected by the subject or caretaker. In this way, subjects are able to be offered or select from Environmental Supplements that are similar in one or more components to the Environmental Supplements deemed to be beneficial for other subjects with similar personal profiles.

In an embodiment, the measured biological data of a subject satisfies a threshold condition for altering the Environmental Supplement based on one or more conditions being satisfied. In an embodiment, the one or more conditions include time (e.g., satisfying a predetermined amount of time, or a request made by the subject due to duration of time), change in subject's status (e.g., health status, alert status, sleep status, or requirement or desire status as determined by the measured biological data (e.g., sensors, biological assays, observations by a healthcare worker, etc.). In an embodiment, when a threshold condition is satisfied, one or more parameters of the Environmental Supplement will be altered as determined by one or more conditions as disclosed. For example, if the subject is nearing a sleep state, one or more alteration in the Environmental Supplement will be made that is conducive to sleep (e.g., shading or dimming of lights, aromatherapy scent extractions in the room (e.g., lavender, chamomile, jasmine, vanilla, etc.), audio alterations (e.g., playing soft music or other soothing sounds, noise canceling, white noise as background, etc.), heating or cooling of the ambient air or the bed/furniture in which the subject is located, and/or other alterations. In an embodiment, the environment is altered dynamically (e.g., visuals of sunlight to dusk to moonlight over time, sound of rain increasing or decreasing). In an embodiment, the environment is altered dynamically in response to sensors monitoring the subject, e.g., detecting that the subject is beginning to fall sleep or measuring the status of the subject's sleep cycle. In an embodiment, the subject is monitored continuously. In an embodiment, the subject is monitored intermittently (e.g. at a predetermined time or a customized time) based on the subject's personal profile or measured biological data. The assistance of a sleep-conducive environment allows the subject to recover more quickly and reduces the rate of re-admission or recurring disease at a time when Medicare has reduced payment to hospitals based on higher than expected readmission rates. See for example, Krumholz, N. Eng. J. Med. 2013, 368:100-102, and Evans, JAMA Int. Med. Healthcare Bus. News, posted Mar. 25, 2013, accessed Mar. 19, 2014, available at the web at modernhealthcare.com/article/20130325/news/303259959, each of which is incorporated herein by reference.

In an embodiment, one or more sensors utilized in monitoring the subject include remote sensors, such as located in at least one of furniture (bed, chair, couch, etc. including seat or armrest parts), walls, floor, door, doorway, pen, computer monitor, mirror, toilet, faucet, door handle, door, TV, TV remote control, etc.

In an embodiment, the one or more sensors include at least one of remote or directly connected sensors, including but not limited to EEG, EKG, respiratory sensors, pupillometer, EOG, MEG, pulsometer, oximeter, fluid detector or analyzer (e.g., interstitial fluid, blood, cerebral spinal fluid, saliva, or other biological fluid analyzer such as a microfluidics device), biomechanical assessor, thermal sensor, spectrophotometer retinal interrogator, respiration detector, spirometer, implanted physiologic sensors, and others disclosed herein.

In an embodiment, as described herein, any of the sensors disclosed may be operably connected with one or more transmitters, receivers, or transceivers.

In an embodiment, the one or more sensors include environmental sensors external to the room for determining outdoor temperature, light, humidity, etc. such that an internal environment may mimic the outdoors or include particular elements thereof.

In an embodiment, the one or more sensors include at least one of ultrasound, bioimpedance, infrared thermometry, audio/visual (e.g., camera), or eye tracking (e.g., imager). See for example, U.S. Pat. App. Pub. Nos. 2010/0049095; 2006/0190419; 2008/0039698; or 2010/0174533, each of which is incorporated herein by reference.

In an embodiment, the Environmental Supplements include a voice-activated and/or voice recognition electronic assistant or web navigator able to search the internet for answers to questions or suggestions for personal habits (e.g., a reminder that it is time to take some medication) can access the subject's personal health records or personal profile (e.g., at the request of the subject or for reminders regarding particular medical regimens), and can also serve to alert a healthcare worker if the subject is not responsive or calls out for help. In an embodiment, the voice-activated and/or voice recognition electronic assistant provides electronic human interaction in any number of possible languages. In an embodiment, the electronic assistant is able to have an intelligent conversation with the subject as output of the system, based on the subject's personal profile (e.g., the assistant may ask how the subject's arthritis is doing today, how the subject is feeling, what the subject is wanting or needing, etc.) and may include personal details that have been input by the subject (e.g., either in conversation or as initial or supplemental data) or a healthcare worker. For example, the electronic assistant might ask about the subject's childhood or happy times in the subject's life if the subject is depressed and would like to discuss such things. In another example, the electronic assistant provides information or intellectual stimulation (e.g., through an audio book, lecture, crossword puzzle or other word/number games, etc.). In an embodiment, the data input by the subject in interacting with the electronic assistant is utilized as part of the dynamic personal profile for determining the Environmental Supplement(s) as well as the overall health of the subject. In an embodiment, the electronic assistant interacts with the subject for discussing menu choices, exercise or physical activity, and may provide encouragement to the subject. In an embodiment, the electronic assistant is digitized as an avatar that can be interacted with by computer, TV, or other monitor, touch screen, personal tablet, smartphone, etc. In an embodiment, the electronic assistant can provide services similar to a human mental health professional particularly with determining the psychological health of the subject or engaging with the subject in such a manner as to coach or guide the subject to better mental health.

In an embodiment, the electronic assistant is pre-programmed for particular dialogue(s) with the subject. In an embodiment, the electronic assistant is pre-programmed to provide dynamic dialogue(s) or “intelligent” and adaptive conversations based on one or more particular algorithms and optionally, customized information particular to a specific subject.

In an embodiment, several Environmental Supplements can be utilized for exercise, including for example, equipment such as a stationary bike, treadmill, elliptical machine, weights, etc., combined with other elements of Environmental Supplements, including customized lighting, dynamic or stereotactic projection, or display of various outdoor or other locales (e.g., the mall, home, etc.) that may be projected or displayed in real-time or with stored (recorded) information, just as disclosed herein for other embodiments. In an embodiment, the exercise equipment includes one or more sensors, including those disclosed herein elsewhere.

As described herein, the subject's personal profile that informs the computing device, which determines the customized Environmental Supplement, is based at least in part on physical aspects of the subject, including but not limited to measurable physical characteristics such as gender or gender identity, height, weight, fingerprint, physical malformations, appearance of skin, hair, or nails, appearance of eyes, respiratory sounds and rate, body temperature, blood gas level, heart rate, brain electrical activity, respiration rate, blood chemistry, blood cell counts, platelet counts, antibody titer, electrolyte levels, blood antigen type, tissue antigen type, evidence of pathogen exposure, lipid levels, perception of pain, depression or mood, body movement, gait, stiffness, tremors, evidence of cognition state, dehydration, injury, malaise, rigor, fever, cough, heart palpitations, numbness or tingling in any part of the body, swelling in any part of the body, wheezing, difficulty swallowing, nasal or bronchial congestion. dizziness or lightheadedness, fainting spells or loss of consciousness, lumps or bumps on the body, dry mouth, nausea, shortness of breath, thirst, weakness, sleepiness, hearing loss or problem, vision loss or problem, constipation, diarrhea, flatulence, other digestive disorder, urinary incontinence, loss of smell or problem, loss of voice or problem, loss of taste or problem, fine motor skill loss or change from prior status, gross motor skill loss or change from prior status, skin changes (e.g., birthmarks, etc.) body odor, content or change in microbiome of any body organ or surface (e.g., mouth, gut, skin, ear, eye, etc.), allergy, infection, addiction, hormonal imbalance, insomnia, or similar characteristics.

In an embodiment, one or more of the physical aspects of the subject utilized in the personal profile may be self-reported, derived from the subject's medical history (e.g., electronic health records), reported by a third party (e.g., insurance company, healthcare worker, family, friend, companion, etc.), or sensed directly in indirectly by the one or more sensors.

In an embodiment, the subject self-reports one or more physical aspects of him/herself by interaction with at least one input/output computing device (e.g., kiosk, tablet, desktop, laptop, handheld device, etc.), through a series of questions or pictures. The subject may also be shown various scenes or locales (e.g., beach, mountain cabin, their own home) and asked which they prefer. In an embodiment, one or more sensors can be used to sense the subject's response to a stimulus, such as a visually provided scene, to determine preference. For example the subject's facial response can be captured and analyzed by an imager such as a camera for pleasant or disturbed response. For example the subject's pupils may be examined by a pupillometer for an autonomic response. For example, the subject's brain functions may be examined by EEG for a neural response.

In an embodiment, one or more sensors (e.g., cameras) are placed throughout the subject's home, or other familiar or unfamiliar environment, with information provided to the systems disclosed herein (for example, sensors outdoors, sensors in the subject's home, in the subject's TV, etc.). The question for preference may come directly or indirectly by asking which provides comfort, which brings peace, which makes the subject feel healthy or happy, or which eases fear or other uncomfortable feelings. Further, a ranking system may be utilized for providing different Environmental Supplements based on how the subject is feeling (e.g., agitated, sleepy, depressed, etc.) or by overall preference regardless of mood or health status.

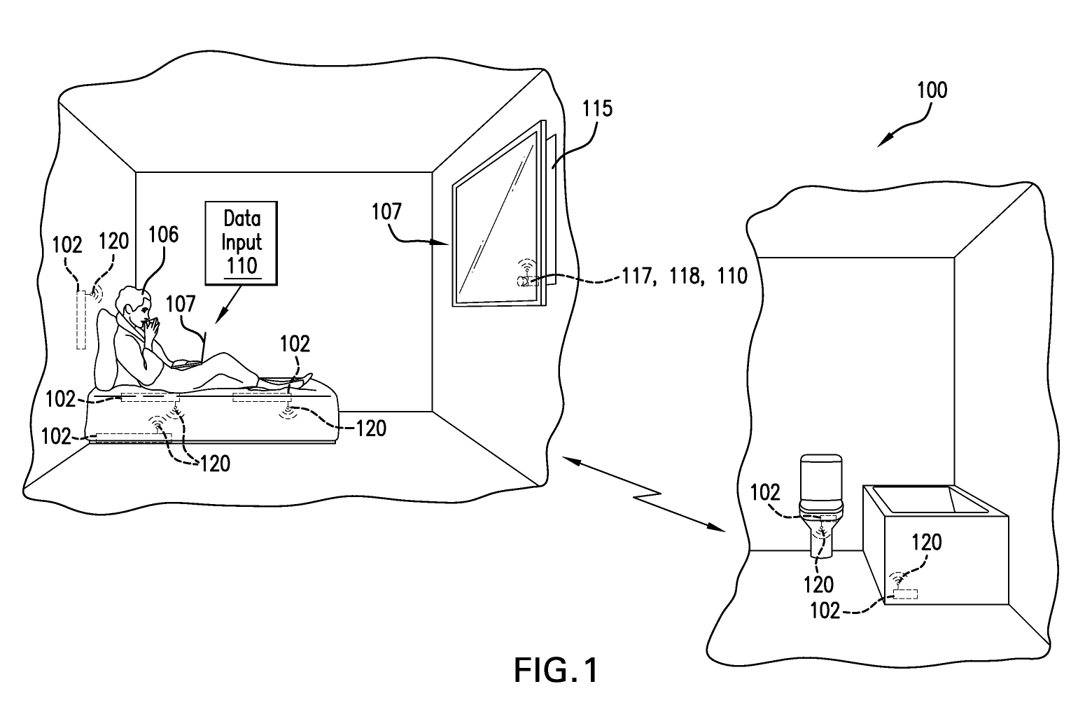

As shown in FIG. 1, the system 100 includes at least one input/output computing device 107 for data input 110 to the system 100 (e.g., a keyboard, mouse or touchpad, speech, or audio/video, not all shown). As indicated, one or more sensors 102 are located in proximity to or in direct contact with the subject 106. As indicated, in an embodiment, the input/output computing device 107 (including a keyboard, audio/video, or other device) may include a receiver 118 (optionally wireless, shown on camera), transceiver 117 (optionally wireless), transmitter 110 (optionally wireless), and may include audio/video capabilities, a power source 115, and memory.

In an embodiment, the system 100 includes circuitry having one or more components operably coupled (e.g., communicatively, electromagnetically, magnetically, ultrasonically, optically, inductively, electrically, capacitively coupled, or the like) to each other. In an embodiment, circuitry includes one or more remotely located components. In an embodiment, remotely located components are operably coupled via wireless communication. In an embodiment, remotely located components are operably coupled via one or more receivers 118, transceivers 117, transmitters 110, or the like.

In an embodiment, circuitry includes, among other things, one or more computing devices such as a processor (e.g., a microprocessor), a central processing unit (CPU), a digital signal processor (DSP), an application-specific integrated circuit (ASIC), a field programmable gate array (FPGA), or the like, or any combinations thereof, and can include discrete digital or analog circuit elements or electronics, or combinations thereof. In an embodiment, circuitry includes one or more ASICs having a plurality of predefined logic components. In an embodiment, circuitry includes one or more FPGA having a plurality of programmable logic components.

In an embodiment, circuitry includes one or more memory devices that, for example, store instructions or data. For example In an embodiment, the system 100 includes one or more memory devices that store information related to one or more characteristics of the subject that has been assessed, electronic health records, self-reported symptoms, insurance, or other health-related information. Non-limiting examples of one or more memory devices include volatile memory (e.g., Random Access Memory (RAM), Dynamic Random Access Memory (DRAM), or the like), non-volatile memory (e.g., Read-Only Memory (ROM), Electrically Erasable Programmable Read-Only Memory (EEPROM), Compact Disc Read-Only Memory (CD-ROM), or the like, persistent memory or the like, Erasable Programmable Read-Only Memory (EPROM), flash memory, or the like. The one or more memory devices can be coupled to, for example, one or more computing devices 107 by one or more instructions, data, or power buses.

In an embodiment, circuitry includes one or more computer-readable media drives, interface sockets, Universal Serial Bus (USB) ports, memory card slots, or the like, and one or more input/output components such as, for example, a graphical user interface, a display, a keyboard, a keypad, a trackball, a joystick, a touch-screen, a mouse, a switch, a dial, or the like, and any other peripheral device. In an embodiment, circuitry includes one or more user input/output components that are operably coupled to at least one computing device to control (electrical, electromechanical, software-implemented, firmware-implemented, or other control, or combinations thereof) at least one parameter associated with, for example, the health information related to the subject's health or status.

In an embodiment, the system is configured to operate in an application service provider format. In an embodiment, the system is configured to be implemented using open source tools. For example, in an embodiment, the system includes using one or more of Java, Java server pages (JSP), Java database connectivity (JDBC), structured query language (SQL), extensible markup language (XML), user interface language (XUL) and/or scalable vector graphics (SVG) technologies.

In an embodiment, image-based applications such as viewers and/or toolkits (e.g., Insight Segmentation and Registration Toolkit (ITK)), are incorporated for further intake of information. In an embodiment, CAD implementations or image segmentation may allow previous processing of images previously accepted on intake of information from the subject.

In an embodiment, circuitry includes a computer-readable media drive or memory slot that is configured to accept non-transitory signal-bearing medium (e.g., computer-readable memory media, computer-readable recording media, or the like). In an embodiment, a program for causing a system to execute any of the disclosed methods can be stored on, for example, a computer-readable recording medium (CRMM), a non-transitory signal-bearing medium, or the like. Non-limiting examples of signal-bearing media include a recordable type medium such as magnetic tape, floppy disk, a hard disk drive, Compact Disc (CD), a Digital Video Disk (DVD), Blu-Ray Disc, digital tape, computer memory, or the like, as well as transmission type medium such as a digital and/or analog communication medium (e.g., fiber optic cable, waveguide, wired communications link, wireless communication link (e.g., receiver 118, transceiver 117, or transmitter 110, transmission logic, reception logic, etc.). Further non-limiting examples of signal-bearing media include, but are not limited to, DVD-ROM, DVD-RAM, DVD+RW, DVD-RW, DVD-R, DVD+R, CD-ROM, Super Audio CD, CD-R, CD+R, CD+RW, CD-RW, Video Compact Discs, Super Video Discs, flash memory, magnetic tape, magneto-optic disk, MINIDISC, non-volatile memory card, EEPROM, optical disk, optical storage, RAM, ROM, system memory, web server, cloud, or the like. In an embodiment, the ADC system 100 includes circuitry having one or more modules optionally operable for communication with one or more input/output components that are configured to relay user output/input. In an embodiment, a module includes one or more instances of electrical, electromechanical, software-implemented, firmware-implemented, or other control devices. Such devices include one or more instances of memory, computing devices, antennas, power or other supplies, logic modules or other signaling modules, gauges or other such active or passive detection components, piezoelectric transducers, shape memory elements, micro-electro-mechanical systems (MEMS) elements, or other actuators.

In an embodiment, the computing device further includes audio/visual input/output connected to the system. In an embodiment, the audio/visual input/output is configured to interact with the subject. In an embodiment, the system further includes a printing device connected to the computing device. In an embodiment, the system includes an input/output device including a graphical interface (e.g., display, touch screen, etc.).

In an embodiment, the one or more sensors include, for example, one or more acoustic sensors, optical sensors, electromagnetic energy sensors, image sensors, photodiode arrays, charge-coupled devices (CCDs), complementary metal-oxide-semiconductor (CMOS) devices, transducers, pressure sensors, optical recognition sensors, infrared sensors, radio frequency component sensors, thermo sensors, three-dimensional sensors (e.g. to assess the subject's facial expressions exhibiting pain or discomfort, flushing or redness, or a subject's gait or other movements, etc.).

In an embodiment, one or more components of the system (e.g., chair or bed, toilet, floor, etc. impregnated with sensors) operate in a networked environment using logic connections to one or more remote computing devices (e.g., a common network node, a network computer, a network node, a peer device, a personal computer, a router, a server, a tablet PC, a tablet, etc.) and typically includes many or all of the elements described above. In an embodiment, the logic connections include connections to a local area network (LAN), wide area network (WAN), and/or other networks. In an embodiment, the logic connections include connections to one or more enterprise-wide computer networks, intranets, and the internet. In an embodiment, the system 100, the one or more components of the system or the like operate in a cloud computing environment including one or more cloud computing systems (e.g., private cloud computing systems, public cloud computing systems, hybrid cloud computing systems, or the like).

In an embodiment the one or more sensors 102 may sense heartbeat intervals or ECG readings remotely by measuring small electrical potentials using a high input impedance electrometer. An example of such a sensor device is described in U.S. Pat. App. Pub. No. 2006/0058694; WO 2003/048789, Harland, Meas. Sci. Technol., supra; Prance, 2007 J. of Physics: Conference Series 76, pp. 1-5, supra, each of which is incorporated herein by reference. Such sensor devices are expected to provide noninvasive and remote monitoring. In an embodiment, the one or more sensors 102 may be worn by the subject in or on clothing or jewelry, such as in wrist bands, or may be in non-conductive contact with the body, for example, as described by U.S. Pat. App. Pub. No. 2006/0058694, supra; WO 2003/048789, supra; C. J. Harland et al., 14 Meas. Sci. Technol. 923-928 (2003), each of which is incorporated herein by reference. In an embodiment, the one or more sensors 102 may be included in or associated with a piece of furniture, such as a chair or desk, or electronics such as a personal computer, or with some other remote item within, e.g., within approximately one meter from the subject. In an embodiment, the one or more sensors 102 are able to measure electric potentials and may be embedded in objects, such as a bed or chair, in direct but non-conductive contact with the subject, for example, as described by U.S. Pat. No. 7,245,956, supra, which is incorporated herein by reference. In an embodiment, the one or more sensors 102 may sense heartbeat intervals and electrocardiographic information by examining physiologic activity of the subject or its organs and may be operable to sense a characteristic of the subject 106 in response to an electromagnetic signal sent at or illuminating the subject and reflected from the subject. In an embodiment, the illuminating may include exposing, subjecting, or directing energy at the subject. Systems using illuminating or reflected electromagnetic signals, including radiofrequency (RF) or microwave signals are described in U.S. Pat. No. 7,272,431; U.S. Pat. App. Pub. No. 2004/0123667; or U.S. Pat. App. Pub. No. 2008/0045832; each of which is incorporated herein by reference. In an embodiment, one or more sensors 102, which may be or include a sensor array, may be deployed, for example, throughout a room, perhaps as part of a smart room network, so as to monitor the subject at rest or in motion, and this may be utilized in determining when/if to change the Environmental Supplement.

In an embodiment, information gathered by the one or more sensors 102 may be communicated to a computer. In an embodiment, information may be communicated to a computer of the system electronically. In an embodiment, information may be communicated to a computer of the system wirelessly, for example using radio waves or ultrasound waves, or Bluetooth™ technology. In an embodiment, a computer may be used to process the information. The computer may be part of a network.

FIG. 1 illustrates one embodiment in which a system 100 includes one or more sensors 102 configured to monitor a subject 106 with regard to the Environmental Supplement. As shown, the subject 106 can be monitored by various modes, including but not limited to, input/output device 107 (e.g., user interface), sensor 102 (e.g., breathalyzer, thermal scan, respiration sensor, pupillometry, retinal scan, weight scale, gait monitor, etc.), one or more of which includes one or more of a receiver 118, transceiver 117, transmitter 110, memory 116, or power source 115. As shown, in an embodiment, an audio or visual criticality indicator 119 signals a subject whose changing physical aspects satisfy a threshold condition (e.g., satisfying an emergency or critical threshold or satisfying a time condition or blood flow threshold), based, for example, on sensors monitoring the subject, self-reporting symptoms, and/or health history records.

In an embodiment, the one or more sensors 102 include a sensor array configured to sense an aspect of the subject's health or status without physically contacting the subject. For example, one sensor head may be configured to sense temperature, another sensor head configured to sense heart rate, and a further sensor head configured to sense blood pressure. In an embodiment, the sensor includes a sensor responsive, without physically contacting the subject, to an impedance, capacitance, permittivity, reflectivity (e.g., acoustic, light, or radiowave reflectivity), absorption, or electrical activity of the subject.

In an embodiment, the sensor may include a sensor configured for a physical association with an object wearable by a subject and to sense a characteristic of the subject without physically contacting the subject. For example, the sensor may be configured for a physical association with eye glasses or jewelry. For example, a sensor configured for a physical association with an object wearable by a subject is described by U.S. Pat. App. Pub. No. 2006/0058694; WO 2003/048789; or C. J. Harland et al., 14 Meas. Sci. Technol. 923-928 (2003), each of which is incorporated herein by reference.

In an embodiment, the sensor device includes an electrodynamic sensor device configured to sense an electrical activity of the heart of a subject without physically contacting the subject. For example, the electrodynamic sensor may be configured to sense a heart rate, electrical activity of the heart, such as electrocardiography (ECG), or conductivity. An example of a high input impedance electrodynamic sensor device configured to sense an electrical activity of a heart of a subject without physically contacting the subject is described in U.S. Pat. App. Pub. No. 2006/0058694 and WO 2003/048789.

In an embodiment, the one or more sensors 102 include a sensor configured to sense at least one of an electrical, acoustic, thermal, radiative, absorption, reflection, gaseous emission, or transmissibility aspect of the subject. In an embodiment, a thermal aspect may include an infrared-measured thermal aspect. In an embodiment, a thermal aspect may include microwave length (3-30 cm) electromagnetic radiation naturally emitted by the subject. For example, a sensor configured to sense a thermal aspect of the subject includes a microwave radiometer operable to measure natural electromagnetic radiation from the subject's internal tissue in the microwave range. In an embodiment, the microwave radiometer may be combined with an infrared sensor as described in R. Avagyan et al., ICMART '99 International Medical Acupuncture Symposium 7, Riga, (May 21-23, 1999), which is incorporated herein by reference. See also, WO 2006/091123 (PCT/RU2006/000072), which is incorporated herein by reference. For example, a transmissibility aspect may include a light or radio wave transmissibility aspect. For example, in an embodiment, a radiative aspect may include gammas or other types of radiation emitted by the body of the subject itself, for example potassium 40. An embodiment of a gamma-ray sensor device configured to sense an aspect of a subject without physically contacting the subject is expected to be provided by the Radtell™ passive gamma-ray sensor by Oak Ridge National Laboratory of Oak Ridge, Tenn.

In an embodiment, a sensor 102 is operably coupled to one or more sensor control units 120 operably coupled to the input/output computing device 107. In an embodiment, the one or more sensor control units 120 serve to regulate the activity of the one or more sensors 102. For example, in an embodiment, one or more sensor control units 120 regulate one or more times when the one or more sensors 102 detect one or more signals from the subject that are related to one or more aspects of the subject. In an embodiment, the one or more sensor control units 120 regulate one or more time periods when one or more sensors 102 detect one or more signals from the subject that are related to one or more aspects of the subject. In an embodiment, one or more sensor control units 120 are operably coupled to one or more detection processors of the input/output computing device 107.

In an embodiment, a sensor 102 is configured to wirelessly communicate sensed electrical signals originating from a subject 106. In an embodiment, a sensor 102 is electrically or optically coupled to the control circuitry to communicate the one or more signals thereto.

In an embodiment, numerous different types of detectors 136 are operably coupled to one or more sensors 102 and are operationally coupled to the input/output computing device. Examples of such detectors 136 include, but are not limited to, electrodes, surface plasmon resonance detectors, microelectromechanical systems detectors, microcantilever detectors, nitric oxide detectors, osmotic detectors, relativity-based detectors, chemical detectors, pressure detectors, electrochemical detectors, piezoelectric detectors, pH detectors, hydrogel detectors, enzymatic detectors, ball integrated circuit detectors, affinity viscosimetric detectors, blood pressure detectors; metal detectors, glucose detectors, and the like (e.g., U.S. Pat. Nos. 7,162,289; 6,280,604; 5,603,820; 5,582,170; 6,287,452; 7,291,503; 6,764,446; 7,168,294; 6,823,717; 7,205,701; 6,268,161; 4,703,756; 6,965,791; 6,546,268; 6,210,326; 6,514,689; 6,234,973; 6,442,413; Tu et al., Electroanalysis, 11:70-74 (1999), each of which is incorporated herein by reference). In an embodiment, one or more detectors 136 are configured to detect one or more of pH, chemicals, or nerve signals from the subject.

In an embodiment, a sensor 102 includes one or more sensor power sources 115 (including but not limited to one or more batteries). In an embodiment, a sensor 102 is operably coupled to one or more sensor batteries 115. In an embodiment, a sensor battery 115 includes a thin-film fuel cell such as a solid oxide type (SOFC), a solid polymer type (SPFC), a proton exchange membrane type (PEMFC), and/or substantially any combination thereof. Methods to fabricate such thin-film fuel cells are known and have been described (e.g., U.S. Pat. No. 7,189,471, incorporated herein by reference). In an embodiment, one or more sensor batteries 115 include one or more storage films that are configured for energy storage and energy conversion. Methods to fabricate such storage films are known and have been described (e.g., U.S. Pat. No. 7,238,628, incorporated herein by reference). In an embodiment, a sensor battery 115 is a biobased battery (e.g., U.S. Pat. No. 6,994,934, incorporated herein by reference). In an embodiment, one or more sensor batteries 115 are thin-film batteries. Methods to fabricate thin-film batteries, including thin film microbatteries, are known and have been described (e.g., U.S. Pat. Nos. 5,338,625; 7,194,801; 7,144,655; 6,818,356, incorporated herein by reference). In an embodiment, one or more sensor electromagnetic receivers (not shown) are used to electromagnetically couple power to energize one or more sensors 102 from an external power source 115. Methods to construct electromagnetic receivers 118 have been described (e.g., U.S. Pat. No. 5,571,152), incorporated herein by reference. In an embodiment, the receiver 118 and/or transmitter 110 are not part of the sensor.

In an embodiment, the system 100 includes one or more sensor transmitters 110. Numerous types of transmitters 110 can be used in association with system 100. Examples of such transmitters 110 include, but are not limited to, transmitters that transmit one or more acoustic signals, optical signals, radio signals, wireless signals, hardwired signals, infrared signals, ultrasonic signals, and the like (e.g., U.S. Pat. Nos. RE39,785; 7,260,768; 7,260,764; 7,260,402; 7,257,327; 7,215,887; 7,218,900), each of which is incorporated herein by reference. In an embodiment, one or more sensor transmitters 110 may transmit one or more signals that are encrypted. Numerous types of transmitters are known and have been described (e.g., U.S. Pat. Nos. 7,236,595; 7,260,155; 7,227,956; and Published U.S. Pat. App. No. US2006/0280307), each of which is incorporated herein by reference.

In an embodiment, the system 100 includes one or more sensor receivers 118. Numerous types of sensor receivers 118 may be used in association with system 100. Examples of such sensor receivers 118 include, but are not limited to, receivers that receive one or more acoustic signals, optical signals, radio signals, wireless signals, hardwired signals, infrared signals, ultrasonic signals, and the like. Such receivers 118 are known and have been described (e.g., U.S. Pat. Nos. RE39,785; 7,218,900; 7,254,160; 7,245,894; 7,206,605), incorporated herein by reference.

In an embodiment, the system 100 includes at least one computing device processor that utilizes at least one set of instructions derived from mathematical trends such as queuing theory. For example, the theory of mathematical queuing allows for derivation and calculation of several performance measures including the average wait time in the queue or the system, the expected number waiting or receiving service, and probability of encountering the system in various states (e.g., empty, full, having an available server or having to wait a certain amount of time to be served). In addition, useful queuing modeling can be based on the Poisson process and its companion exponential probability distribution, which mimics the response of the system being modeled to those same inputs.

Various statistical programs or computer algorithms for simulating systems may be implemented with various embodiments described herein. For example, ANOVA, Monte Carlo, etc., and other programs may be implemented.

In an embodiment, the Environmental Supplement includes at least one customized setting including temperature, lighting (light/dark schedule as well as light intensity in the room at a given time), humidity, vibration (e.g., of a bed, chair, crib, bassinet, etc.), influx of fresh air from outdoors or filtered air, images (e.g., stored or generated real-time) of various locales (e.g., the subject's own home, the subject's own childhood home or historical travels, internet images, etc.) or individuals (the subject's friends or family or pets), or outdoor scenes (e.g., ocean, beach, mountains, snow, golf course, rain, sunshine, fields, flowers, city street, farmer's market, rural area, etc.), relative situation (e.g., the ceiling may reflect what the sky above it looks like to give the sense of being outdoors and of connectedness), or sounds (e.g., birds singing, ocean waves, rain falling, wind blowing, cars in the street, heartbeat, breathing, children talking or laughing, animal sounds, music, etc.).

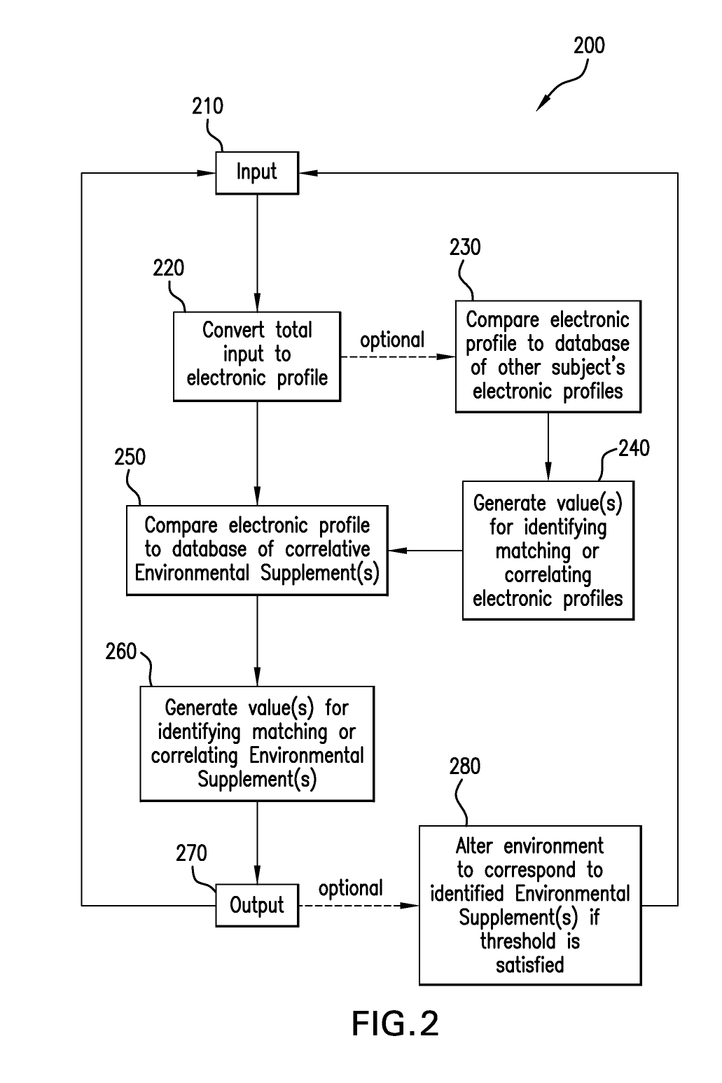

As described in FIG. 2, a personal profile can be built and modified in various ways. In an embodiment, the subject's personal profile is compared with other subjects' profiles in a dataset in order to offer or suggest corresponding Environmental Supplements that have benefited other subjects with similar profiles or similar health conditions. In an embodiment, the dataset is a static data structure. In an embodiment, the dataset is a dynamic data structure.

As shown at 200, the system and method include an input 210 which may be verbal input from the subject or a healthcare worker; electronic input from the subject, a healthcare worker, or another computer; sensed input from one or more sensors; or input from electronic health record or other information transferred by computer, etc. as disclosed herein. The cumulative input information is converted into the electronic personal profile 220. An optional step includes comparing the subject's personal profile to a database of other subjects' personal electronic profiles 230. From this comparison, one or more values are generated for identifying matching or correlating electronic profiles 240. In the absence of or in addition to comparing the subject's personal profile with the database of second personal electronic profiles, the subject's personal profile is compared to a database of correlative Environmental Supplement(s) 250 to generate a menu of offerings or suggestions deemed to be beneficial for the overall health of the subject, or for the specific addressing of a particular ailment (e.g., depression, aching muscles, etc.). From this comparison, one or more values are generated for identifying matching or correlating the specific Environmental Supplement(s) 260 deemed to be most beneficial to this particular subject, based on one or more algorithms and one or more databases. The conclusion for the round of determining Environmental Supplement(s) is produced as output 270 from the system. As this is a loop, the round may be the primary round, secondary round, tertiary round, etc. In an embodiment, a component of the system and method involves electronically instructing or causing an alteration 280 to the subject's environment (e.g., alter lighting, sounds, visual images, electronic assistant communication, etc.) based on the identification of the Environmental Supplement(s). In an embodiment, a threshold condition must be satisfied in order for the system and method to instruct the alteration of the subject's environment. As described herein elsewhere, the output includes, but is not limited to, suggestions or choices of various Environmental Supplements, including visual, audio, recorded information, real-time information, computer-generated information, electronic assistant(s) or others described herein. Various audio or visual devices for providing the Environmental Supplement(s) have been described herein, including for example, speakers (e.g., to provide white noise, music, or sounds, and may include surround sound or pan sound), sound dampening devices (e.g., retractable, noise-dampening wall or window treatments, automatic screens surrounding the subject such as one in a bed or chair), projectors (e.g., 2D, 3D, holographic, etc.), television(s) as one or an array, dynamic projection, stereoscope projection, etc. and may include one or more projectors including those that are capable of changing the surface topography or geometry in the room, rear projection screens, high resolution screens, gigapixel screens (LED, LCD, etc.), powerwalls, tiled or modular displays, immersive visualization displays, spatially immersive displays, immersive computer graphics, monitors (e.g., closed circuit monitors with cameras set at home for a subject's ability to ascertain, and enjoy and partake in, the home environment such as pets, household events, etc.), dimmable machine information displays, retractable or movable screens, etc. In an embodiment, the audio delivery device includes at least one of a sound dampening, white noise delivery, or noise-cancellation device. In an embodiment, the audio delivery device includes at least one device for producing at least one of surround sound, pan sound, or spatial sound. Example of systems for producing pan and spatial sound are the Sound Element Spatializer (ICMC Vol. 2011) on the worldwide web at mat.ucsb.edu/Publications/McGee ICMC 2001.pdf, and the ‘Spatial Pan’ sound system from the Fraunhofer Institute for Digital Media Technology, which is part of the Immersive Dome.

In an embodiment, the visual display device includes at least one monitor, projector, immersive visualization display, dimmable machine information display, or projection screen. In an embodiment the visual display device includes at least one of a 2-dimensional, 3-dimensional, holographic, forward projecting, rear projecting, illumination, immersive, dynamic projection, stereoscopic projection, or surface-changing technology. In an embodiment the visual display device includes at least one of a rear projection screen, a high resolution screen, a gigapixel screen, an LED display or an LCD display. In an embodiment the visual display device includes at least one of a modular, array, or tiled screen or display. In an embodiment the visual display device includes a powerwall. In an embodiment, the visual display device comprises a computer assisted virtual environment (CAVE) or an immersive virtual reality environment. Examples of visual display devices and immersive environments include those described by Sukthankar CVIIE (2005), pp. 162-172 Towards Ambient Projection for Intelligent Environments; Parke (Proceedings Sigradi 2002, pp. 163-166, 27-28 Nov. 2002 “Next Generation Immersive Visualization Environments”), and Yapo (Computer Vision and Pattern Recognition Workshops (CVPRW), pages 1-8, Dynamic projection environments for immersive visualization 1993), Peterka, et al., Advances in the Dynallax Solid-State Dynamic Parallax Barrier Autostereoscopic Visualization Display System, IEEE Transactions on Visualization and Computer Graphics, Vol. 14, No. 3, pp. 487-499, May/June 2008, on mcs.anl.gov/˜tpeterka/immerse.html, accessed Mar. 31, 2014; and in U.S. Pat. No. 7,636,365, Smart digital modules and smart digital wall surfaces combining the same, and context aware interactive multimedia system using the same and operation method thereof, to Chang. Additional examples of visual displays include the Stony Brook Reality Deck and the Fraunhofer Institute Immersive Dome.

In an embodiment the visual display device includes at least one projector. In an embodiment the projector includes at least one of a forward projecting, rear projecting, illumination, dynamic projection, stereoscopic projection, or surface-changing projector. In an embodiment, the at least one projector can project onto a static or planar surface. In an embodiment the projector can project onto a nonplanar surface or nonstatic surface, for example when information regarding the nonplanar or nonstatic surface is ascertained by a sensor (e.g., a camera or imager scanning structured light patterns, which may include tracking) For example, the projector can project onto at least a portion of a wall, a ceiling, a floor, an article of furniture, or equipment. In an embodiment the projector can project onto a screen, for example a screen built into a wall or furniture.

In an embodiment the visual display device includes at least one screen. In an embodiment, the screen includes at least one of a retractable or movable screen. In an embodiment, the screen includes a projection screen. In an embodiment, the screen includes a light-blocking screen. In an embodiment, the screen includes a noise-dampening or noise-canceling screen. In an embodiment, the screen includes at least one of wheels, motors, mechanisms, transmitters, receivers, and circuitry. In an embodiment, the screen includes an audio device or a video device. In an embodiment, the screen can transmit, receive, or reflect images, sounds, or other supplements. In an embodiment, the screen is at least one of a retractable, track-run, locatable, or stationary screen. In an embodiment, the stationary screen is built into a wall or furniture of the subject's setting. In an embodiment, the retractable screen is at least one of telescoping, accordion, or flap screen. In an embodiment, the screen positioning or output is controlled by the computing device.

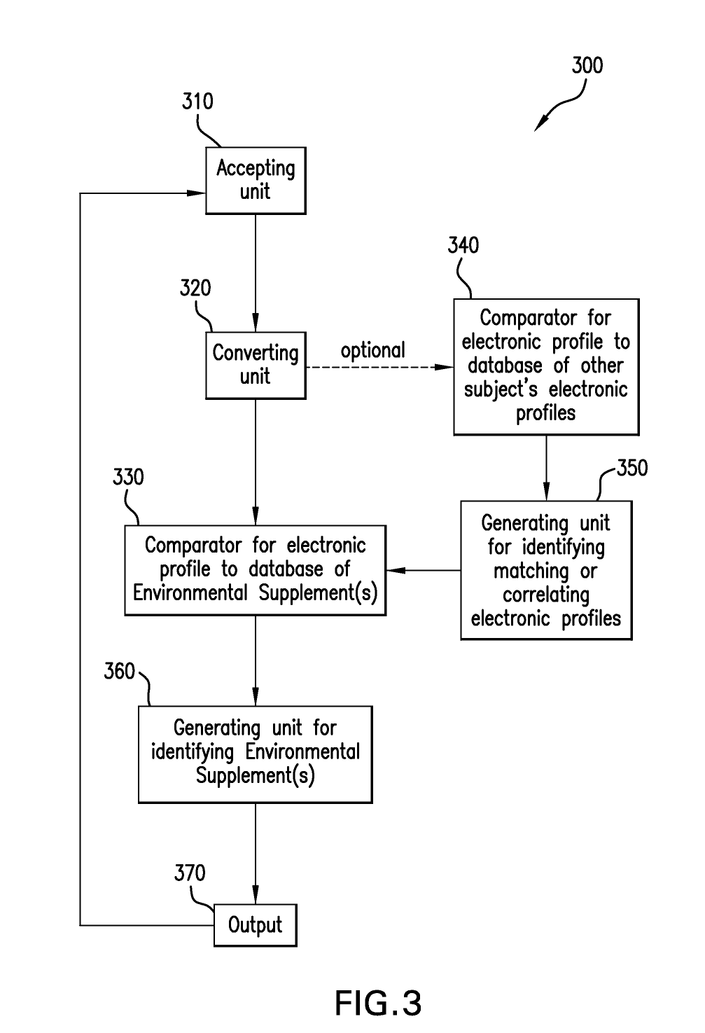

As shown in FIG. 3 at 300, the system includes various components that are operably connected electronically, including an accepting unit 310 for receiving input as described in FIG. 2, a converting unit 320 for converting the raw data into a personal profile that represents the subject's information related to health and personal behavior, an optional comparator 340 for comparing the personal profile with a database of other subjects' personal profiles, an optional generating unit 350 for identifying or matching this particular queried subject's personal profile with others in the database, a comparator 330 for comparing the subject's personal profile with the database of Environmental Supplement(s), a generating unit 360 for identifying the matching or correlative Environmental Supplement(s), and output 370 that represents the determination of the beneficial Environmental Supplement(s) that are either suggested for the subject, or offered to the subject from which to choose. In an embodiment, the output 370 is the instruction or action of the automated system based on the comparison, without any choice provided to the subject such that the system is fully automated based on the detected data from the one or more sensors associated with the subject, in the room, or external to the room in which the subject is located (e.g., outdoors).

In an embodiment, the comparator, or comparing step, includes instructing a computing device to retrieve from storage one or more parameters associated with at least one Environmental Supplement and to perform a comparison of at least one input (e.g., detected from one or more sensors, information from the subject's personal health history, camera data, etc.) to the retrieved one or more parameters. In an embodiment, the transceiver and/or transmitter and/or receiver are operably linked to the comparator to concurrently or sequentially transmit or receive information in response to the comparison.

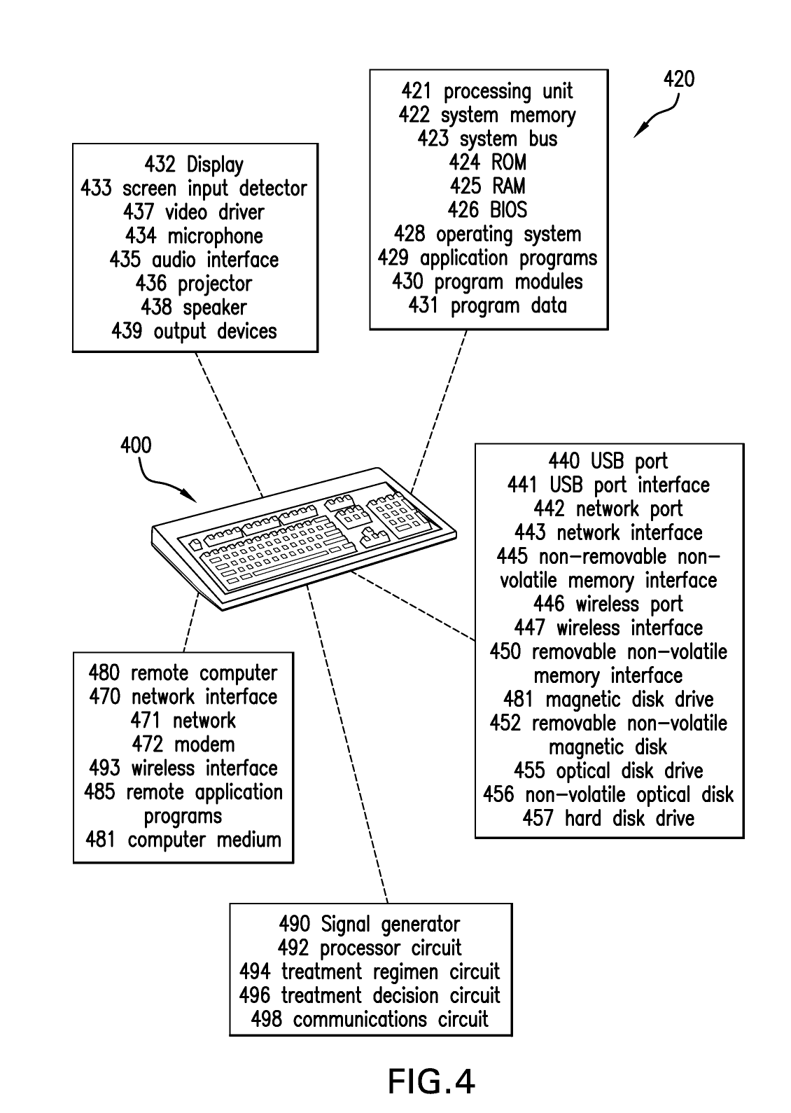

FIG. 4 illustrates an input/output device 400 operably coupled with a computing device 420 that includes a processing unit 421, a system memory 422, and a system bus 423 that couples various system components including the system memory 422 to the processing unit 421. The system bus 423 may be any of several types of bus structures including a memory bus or memory controller, a peripheral bus, and a local bus using any of a variety of bus architectures. The system bus 423 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus. The system memory includes read-only memory (ROM) 424 and random access memory (RAM) 425. A basic input/output system (BIOS) 426, containing the basic routines that help to transfer information between sub-components within the thin computing device 420, such as during start-up, is stored in the ROM 424. A number of program modules may be stored in the ROM 424 or RAM 425, including an operating system 428, one or more application programs 429, other program modules 430 and program data 431.

A user may enter commands and information into the computing device 420 through input devices, such as a number of switches and buttons, illustrated as hardware buttons 444, connected to the system via a suitable interface 445. Input devices may further include a touch-sensitive display with suitable input detection circuitry, illustrated as a display 432 and screen input detector 433. The output circuitry of the touch-sensitive display 432 is connected to the system bus 423 via a video driver 437. Other input devices may include a microphone 434 connected through a suitable audio interface 435, and a physical hardware keyboard (not shown). Output devices may include at least one the display 432, or a projector display 436.

In addition to the display 432, the computing device 420 may include other peripheral output devices, such as at least one speaker 438. Other external input or output devices 439, such as a joystick, game pad, satellite dish, scanner or the like may be connected to the processing unit 421 through a USB port 440 and USB port interface 441, to the system bus 423. Alternatively, the other external input and output devices 439 may be connected by other interfaces, such as a parallel port, game port or other port. The computing device 420 may further include or be capable of connecting to a flash card memory (not shown) through an appropriate connection port (not shown). The computing device 420 may further include or be capable of connecting with a network through a network port 442 and network interface 443, and through wireless port 446 and corresponding wireless interface 447 may be provided to facilitate communication with other peripheral devices, including other computers, printers, and so on (not shown). It will be appreciated that the various components and connections shown are examples and other components and means of establishing communication links may be used. Thus, in an embodiment, the output may include paper or computer read out (e.g., display) information, or may include instructions within the automated system to alter the environment as determined by the comparison. In this way, the dynamic system includes output that includes a sensory alteration (e.g., alteration in visual or audio display, alteration in temperature or humidity of the room, alteration of one or more walls of the room that include images, etc.).

In an embodiment, the output is communicated to a user of the system. In an embodiment, the user may include the subject itself or may include a health care worker or a second computer or computer system.

The computing device 420 may be designed to include a user interface. The user interface may include a character, a key-based, or another user data input via the touch sensitive display 432. The user interface may include using a stylus (not shown). Moreover, the user interface is not limited to an actual touch-sensitive panel arranged for directly receiving input, but may alternatively or in addition respond to another input device such as the microphone 434. For example, spoken words may be received at the microphone 434 and recognized. Alternatively, the computing device 420 may be designed to include a user interface having a physical keyboard (not shown).

In certain instances, one or more components of the computing device 420 may be deemed not necessary and omitted. In other instances, one or more other components may be deemed necessary and added to the computing device.

In certain instances, the computing system typically includes a variety of computer-readable media products. Computer-readable media may include any media that can be accessed by the computing device 420 and include both volatile and nonvolatile media, removable and non-removable media. By way of example, and not of limitation, computer-readable media may include computer storage media. By way of further example, and not of limitation, computer-readable media may include a communication media.

Computer storage media includes volatile and nonvolatile, removable and non-removable media implemented in any method or technology for storage of information such as computer-readable instructions, data structures, program modules, or other data. Computer storage media includes, but is not limited to, random-access memory (RAM), read-only memory (ROM), electrically erasable programmable read-only memory (EEPROM), flash memory, or other memory technology, CD-ROM, digital versatile disks (DVD), or other optical disk storage, magnetic cassettes, magnetic tape, magnetic disk storage, or other magnetic storage devices, or any other medium which can be used to store the desired information and which can be accessed by the computing device 420. In a further embodiment, a computer storage media may include a group of computer storage media devices. In another embodiment, a computer storage media may include an information store. In another embodiment, an information store may include a quantum memory, a photonic quantum memory, or atomic quantum memory. Combinations of any of the above may also be included within the scope of computer-readable media.

Communication media may typically embody computer-readable instructions, data structures, program modules, or other data in a modulated data signal such as a carrier wave or other transport mechanism and include any information delivery media. The term “modulated data signal” means a signal that has one or more of its characteristics set or changed in such a manner as to encode information in the signal. By way of example, and not limitation, communication media include wired media, such as a wired network and a direct-wired connection, and wireless media such as acoustic, RF, optical, and infrared media.

The computing device 420 may also include other removable/non-removable, volatile/nonvolatile computer storage media products. For example, such media includes a non-removable non-volatile memory interface (hard disk interface) 445 reads from and writes for example to non-removable, non-volatile magnetic media, or a removable non-volatile memory interface 450 that, for example, is coupled to a magnetic disk drive 451 that reads from and writes to a removable, non-volatile magnetic disk 452, or is coupled to an optical disk drive 455 that reads from and writes to a removable, non-volatile optical disk 456, such as a CD ROM. Other removable/nonremovable, volatile/non-volatile computer storage media that can be used in the example operating environment include, but are not limited to, magnetic tape cassettes, memory cards, flash memory cards, DVDs, digital video tape, solid state RAM, and solid state ROM. The hard disk drive 457 is typically connected to the system bus 423 through a non-removable memory interface, such as the interface 445, and magnetic disk drive 451 and optical disk drive 455 are typically connected to the system bus 423 by a removable non-volatile memory interface, such as interface 450.

The drives and their associated computer storage media discussed above provide storage of computer-readable instructions, data structures, program modules, and other data for the computing device 420.

A user may enter commands and information into the computing device 420 through input devices such as a microphone, keyboard, or pointing device, commonly referred to as a mouse, trackball, or touch pad. Other input devices (not shown) may include at least one of a touch sensitive display, joystick, game pad, satellite dish, and scanner. These and other input devices are often connected to the processing unit through a user input interface that is coupled to the system bus, but may be connected by other interface and bus structures, such as a parallel port, game port, or a universal serial bus (USB).

The computing system may operate in a networked environment using logical connections to one or more remote computers, such as a remote computer 480. The remote computer 480 may be a personal computer, a server, a router, a network PC, a peer device, or other common network node, and typically includes many or all of the elements described above relative to the computing device 420, although only a memory storage device. The network logical connections include a local area network (LAN) and a wide area network (WAN), and may also include other networks such as a personal area network (PAN) (not shown). Such networking environments are commonplace in offices, enterprise-wide computer networks, intranets, and the Internet.

When used in a networking environment, the computing system is connected to the network 471 through a network interface, such as the network interface 470, the modem 472, or the wireless interface 493. The network may include a LAN network environment, or a WAN network environment, such as the Internet. In a networked environment, program modules depicted relative to the computing device 420, or portions thereof, may be stored in a remote memory storage device. By way of example, and not limitation, remote application programs 485 as residing on computer medium 481. It will be appreciated that the network connections shown are examples and other means of establishing communication link between the computers may be used.

In certain instances, one or more elements of the computing device 420 may be deemed not necessary and omitted. In other instances, one or more other components may be deemed necessary and added to the computing device 420.

The signal generator 490 includes a signal generator configured to generate a signal indicative of the sensed characteristic of the subject. In one embodiment, the signal may include a raw data signal, i.e., a capacitance measurement, a change in position of skin over artery in the neck, an acoustic pressure, or a brain electrical activity of the subject. In one embodiment, the signal generator may include a processor circuit 492, a treatment regimen circuit 494, a treatment decision circuit 496, or a communications circuit 498. In one embodiment, the communications circuit may be operable to communicate using an electrical conductor or using a wireless transmission. In one embodiment, the signal generator may include an instance of the thin computing device 420 and the processor circuit may be the processing unit 421.

In one embodiment, the system actively monitors (e.g., detects, tracks, etc.) a subject located by using at least one of computerized axial tomography, fiber optic thermometry, infrared thermography, magnetic resonance imaging, magnetic resonance spectroscopy, microwave thermography, microwave dielectric spectroscopy, positron emission tomography, ultrasound reflectometry, spectroscopic imaging, visual imaging, infrared imaging, single photon emission computed tomography, electronic nose, or the like.

In one embodiment, the system includes a subject-tracking system (not shown in figures). For example, in one embodiment, the system includes a subject-tracking system for updating in real time a subject's virtual location in a virtual space corresponding to the physical location of the subject in a physical space, such as a healthcare facility, inpatient room, or outpatient procedure room. In one embodiment, the subject-tracking system includes an optical recognition distributed sensor network that generates Criticality Value based in part on the continuous monitoring of the overall physical condition of the subject, including subject's movements, gait, etc.

All of the above U.S. patents, U.S. patent application publications, U.S. patent applications, foreign patents, foreign patent applications and non-patent publications referred to in this specification and/or listed in any Application Data Sheet, are incorporated herein by reference, to the extent not inconsistent herewith. Those having skill in the art will recognize that the state of the art has progressed to the point where there is little distinction left between hardware, software, and/or firmware implementations of aspects of systems; the use of hardware, software, and/or firmware is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

In some implementations described herein, logic and similar implementations may include software or other control structures suitable to operation. Electronic circuitry, for example, may manifest one or more paths of electrical current constructed and arranged to implement various logic functions as described herein. In some implementations, one or more media are configured to bear a device-detectable implementation if such media hold or transmit a special-purpose device instruction set operable to perform as described herein. In some variants, for example, this may manifest as an update or other modification of existing software or firmware, or of gate arrays or other programmable hardware, such as by performing a reception of or a transmission of one or more instructions in relation to one or more operations described herein. Alternatively or additionally, in some variants, an implementation may include special-purpose hardware, software, firmware components, and/or general-purpose components executing or otherwise invoking special-purpose components. Specifications or other implementations may be transmitted by one or more instances of tangible transmission media as described herein, optionally by packet transmission or otherwise by passing through distributed media at various times.

Alternatively or additionally, implementations may include executing a special-purpose instruction sequence or otherwise invoking circuitry for enabling, triggering, coordinating, requesting, or otherwise causing one or more occurrences of any functional operations described above. In some variants, operational or other logical descriptions herein may be expressed directly as source code and compiled or otherwise invoked as an executable instruction sequence. In some contexts, for example, C++ or other code sequences can be compiled directly or otherwise implemented in high-level descriptor languages (e.g., a logic-synthesizable language, a hardware description language, a hardware design simulation, and/or other such similar mode(s) of expression). Alternatively or additionally, some or all of the logical expression may be manifested as a Verilog-type hardware description or other circuitry model before physical implementation in hardware, especially for basic operations or timing-critical applications. Those skilled in the art will recognize how to obtain, configure, and optimize suitable transmission or computational elements, material supplies, actuators, or other common structures in light of these teachings.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, subjectively and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link (e.g., transmitter, receiver, transmission logic, reception logic), etc.).

In a general sense, those skilled in the art will recognize that the various embodiments described herein can be implemented, subjectively and/or collectively, by various types of electro-mechanical systems having a wide range of electrical components such as hardware, software, firmware, and/or virtually any combination thereof and a wide range of components that may impart mechanical force or motion such as rigid bodies, spring or torsional bodies, hydraulics, electro-magnetically actuated devices, and/or virtually any combination thereof. Consequently, as used herein “electro-mechanical system” includes, but is not limited to, electrical circuitry operably coupled with a transducer (e.g., an actuator, a motor, a piezoelectric crystal, a Micro Electro Mechanical System (MEMS), etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), electrical circuitry forming a communications device (e.g., a modem, communications switch, optical-electrical equipment, etc.), and/or any non-electrical analog thereto, such as optical or other analogs. Those skilled in the art will also appreciate that examples of electro-mechanical systems include but are not limited to a variety of consumer electronics systems, medical devices, as well as other systems such as motorized transport systems, factory automation systems, security systems, and/or communication/computing systems. Those skilled in the art will recognize that electro-mechanical as used herein is not necessarily limited to a system that has both electrical and mechanical actuation except as context may dictate otherwise.

In a general sense, those skilled in the art will recognize that the various aspects described herein which can be implemented, subjectively and/or collectively, by a wide range of hardware, software, firmware, and/or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), and/or electrical circuitry forming a communications device (e.g., a modem, communications switch, optical-electrical equipment, etc.). Those having skill in the art will recognize that the subject matter described herein may be implemented in an analog or digital fashion or some combination thereof.

Those skilled in the art will recognize that at least a portion of the devices and/or processes described herein can be integrated into an image processing system. Those having skill in the art will recognize that a typical image processing system generally includes one or more of a system unit housing, a video display device, memory such as volatile or non-volatile memory, processors such as microprocessors or digital signal processors, computational entities such as operating systems, drivers, applications programs, one or more interaction devices (e.g., a touch pad, a touch screen, an antenna, etc.), control systems including feedback loops and control motors (e.g., feedback for sensing lens position and/or velocity; control motors for moving/distorting lenses to give desired focuses). An image processing system may be implemented utilizing suitable commercially available components, such as those typically found in digital still systems and/or digital motion systems.

Those skilled in the art will recognize that at least a portion of the devices and/or processes described herein can be integrated into a data processing system. Those having skill in the art will recognize that a data processing system generally includes one or more of a system unit housing, a video display device, memory such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), and/or control systems including feedback loops and control motors (e.g., feedback for sensing position and/or velocity; control motors for moving and/or adjusting components and/or quantities). A data processing system may be implemented utilizing suitable commercially available components, such as those typically found in data computing/communication and/or network computing/communication systems.

Those skilled in the art will recognize that at least a portion of the devices and/or processes described herein can be integrated into a mote system. Those having skill in the art will recognize that a typical mote system generally includes one or more memories such as volatile or non-volatile memories, processors such as microprocessors or digital signal processors, computational entities such as operating systems, user interfaces, drivers, sensors, actuators, applications programs, one or more interaction devices (e.g., an antenna USB ports, acoustic ports, etc.), control systems including feedback loops and control motors (e.g., feedback for sensing or estimating position and/or velocity; control motors for moving and/or adjusting components and/or quantities). A mote system may be implemented utilizing suitable components, such as those found in mote computing/communication systems. Specific examples of such components entail such as Intel Corporation's and/or Crossbow Corporation's mote components and supporting hardware, software, and/or firmware.

Those skilled in the art will recognize that it is common within the art to implement devices and/or processes and/or systems, and thereafter use engineering and/or other practices to integrate such implemented devices and/or processes and/or systems into more comprehensive devices and/or processes and/or systems. That is, at least a portion of the devices and/or processes and/or systems described herein can be integrated into other devices and/or processes and/or systems via a reasonable amount of experimentation. Those having skill in the art will recognize that examples of such other devices and/or processes and/or systems might include—as appropriate to context and application—all or part of devices and/or processes and/or systems of (a) an air conveyance (e.g., an airplane, rocket, helicopter, etc.), (b) a ground conveyance (e.g., a car, truck, locomotive, tank, armored personnel carrier, etc.), (c) a building (e.g., a home, warehouse, office, etc.), (d) an appliance (e.g., a refrigerator, a washing machine, a dryer, etc.), (e) a communications system (e.g., a networked system, a telephone system, a Voice over IP system, etc.), (f) a business entity (e.g., an Internet Service Provider (ISP) entity such as Comcast Cable, Qwest, Southwestern Bell, etc.), or (g) a wired/wireless services entity (e.g., Sprint, Cingular, Nextel), etc.

In certain cases, use of a system or method may occur in a territory even if components are located outside the territory. For example, in a distributed computing context, use of a distributed computing system may occur in a territory even though parts of the system may be located outside of the territory (e.g., relay, server, processor, signal-bearing medium, transmitting computer, receiving computer, etc. located outside the territory). A sale of a system or method may likewise occur in a territory even if components of the system or method are located and/or used outside the territory.

Further, implementation of at least part of a system for performing a method in one territory does not preclude use of the system in another territory.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

Those skilled in the art will appreciate that a user may be representative of a human user, a robotic user (e.g., computational entity), and/or substantially any combination thereof (e.g., a user may be assisted by one or more robotic agents) unless context dictates otherwise.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “operably coupled to” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

In some instances, one or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

Various non-limiting embodiments are described herein as Prophetic Examples.

### Prophetic Examples 1

### Prophetic Example 1

### Responsive Dynamic Environment System for a Hospital Room

A computer-controlled system is adapted for use in a hospital room to alter an environment based on personal profile data obtained from a patient in order to promote wellness during and after a stay in the hospital. The system is designed to provide comfort and stimulation to the patient and thereby promote healthy cognition, as well as prevent post-hospital syndrome (see e.g., Krumholz, N. Engl. J. Med. 2013; 368:100-102, which is incorporated herein by reference) and reduce the likelihood of readmission. The system responds to input from physiologic and environmental sensors by providing audio, visual, and environmental supplement to dynamically change the patient's environment and promote wellness based on a comparison with the biological data in light of the patient's profile data and the environmental options available.

Physiologic and medical are deployed to monitor the patient's status. Physiologic and medical sensors are placed on the patient or in the hospital room and transmit data to the system computer (server). For example, remote electroencephalograph (EEG) detectors are used to monitor and report brain electrical activity including beta, alpha, theta, and delta waves associated with waking, relaxation, light sleep and heavy sleep respectively (see e.g., Harland et al., Applied Physics Letters 81: 3284-3286, 2002, which is incorporated herein by reference). A heart rate monitor is placed on the patient's chest to wirelessly transmit heart rate data to the system computer (e.g., eMotion ECG Mobile available from Mega, Kuopio, Finland), and a sleep monitor (e.g., Fitbit Force™ available from Fitbit, Inc., San Francisco, Calif.; or the Actiwatch actigraph from Philips, Andover, Mass.) is placed on the patient's wrist. The sensors connect by wireless communication to the system computer and continuously transmit physiologic data, which is analyzed by the system. For example, EEG data, heart rate data, and motion data may indicate a stage of sleep (e.g., stage 1 theta wave sleep or stage 4 delta wave sleep). See for example, NIH info sheet: Brain Basics: Understanding Sleep available online at the worldwide web at ninds.nih.gov/disorders/brain basics/understanding_sleep.htm, the content of which is incorporated herein by reference.

The system includes additional sensors to monitor the patient's nutrition and health. For example, an electronic nose is installed near the patient's nose and mouth to detect chemicals and biomolecules that are biomarkers for the health status of the patient. An electronic nose with conductive-polymer sensors and pattern recognition hardware (see e.g., Chiu et al., Sensors 13: 14214-14247, 2013, which is incorporated herein by reference) is used to detect biomarkers indicating bacterial infections, respiratory disease, histidinemia, hyperglycemia, ketosis, oxidative stress, diabetes, allograft rejection, carbon dioxide, and oxygen or volatile organic compounds (see e.g., Wilson et al., Sensors 11: 1105-1176, 2011; Turner et al., 2013, J. Breath Res., Vol. 7, No. 1, Abstract, each of which is incorporated herein by reference). In another example, electronic nose sensors may be placed near the subject's face (e.g., in a portion of a bed or chair that is near the subject's face) for detection of volatile organic compounds, as well as other health-related compounds. See for example, U.S. Pat. App. Pub. No. 2012/0011918, and U.S. Pat. Nos. 6,631,333; 8,052,611; and 7,122,152; each of which is incorporated herein by reference. Levels of volatile organic compounds can indicate how long one has been asleep. See, e.g., King et al., Physiol Meas., 2012 March; 33(3):413-428, Abstract, Measurement of endogenous acetone and isoprene in exhaled breath during sleep, which is incorporated herein by reference.

Room sensors are deployed to monitor the patient's status and to monitor internal environments. Environmental sensors are employed to provide input on the patient's room. A thermometer, hygrometer, light sensor, and noise sensors are placed within the room to measure the status of environmental parameters. Sensors are deployed in the hospital room to report temperature, light levels, noise levels, humidity, and oxygen content to the system computer. A video camera focused on the subject may be used to monitor the patient's health status. For example, patient movements in and out of bed, food consumption, sleep/wake times, gait and mannerisms (tremors, dizziness, imbalance, etc.), and facial expressions are monitored to assess the health status of the patient. Video data may also indicate boredom or anxiety, and may document the medication schedule, the hours of sleep, and the frequency and number of nurse and physician visits. Video data is analyzed by the computer to identify patient movements, facial expressions, behaviors and basic living activities, e.g., eating, bathroom, ambulation and sleeping. Computer systems and methods to analyze video images and report activities, events, and emotions can be adapted for use with the system described herein (see e.g., U.S. Pat. No. 7,307,543, and U.S. Pat. No. 7,999,857, each of which are incorporated herein by reference). Moreover, audiovisual data can be analyzed to monitor the hospital room environment, e.g., lighting and noise level, and can include tracking capabilities as well, to monitor the patient's location within the room.

Environmental sensors are deployed or accessed to monitor external environments. Environmental sensors are placed at a distal site (e.g., outside the facility, outdoors in a park, or at the patient's home) to transmit data to the system computer (server). Environmental sensors are employed to provide input on external/distal environments. Sensors are deployed at a distal site to report temperature, light levels, noise levels, humidity, and oxygen content to the system computer. For example, a temperature sensor, a light sensor, and atmospheric sensors may be deployed at a park near the hospital to monitor and report the environmental parameters outdoors. Environmental sensors to measure air quality, humidity, temperature and ambient light are available from Digi-Key Corp., Thief River Falls, Minn.). Environmental data input is transmitted wirelessly over the internet to the system computer.

In response to input from the physiological, medical and environmental sensors monitoring the patient and the patient's room (see above), the computer-controlled system delivers audio, visual, and environmental supplement to the patient. The system provides an immersive environment to reduce stress and anxiety, promote sufficient sleep, encourage physical activity and proper nutrition, promote social interaction, and enhance cognitive functioning. For example in response to sensor input (see above) indicating the patient is anxious or tense, the computer-controlled system creates an immersive environment using audiovisual display (see e.g., Sukthankar CVIIE (2005), pp. 162-172 Towards Ambient Projection for Intelligent Environments; Parke (Proceedings Sigradi 2002, pp. 163-166, 27-28 Nov. 2002 “Next Generation Immersive Visualization Environments”), which is incorporated herein by reference). In response to anxiety or other biological data or measured data sensed by the system, the environmental supplement can be altered, such as with video of the patient's home environment transmitted and reproduced on audiovisual displays to provide familiar environmental cues. Home environmental cues including aromas, lighting, wind and weather sounds, music, and familiar television shows are reproduced in the patient's hospital room to reduce stress and anxiety and promote sleep. Audiovisual output to reproduce the patient's home environment in the hospital may be done with bidirectional audio/video communication systems which incorporate additional channels to transmit environmental information (see e.g., U.S. Pat. No. 8,520,050 Ibid.).

Alternatively, in response to sensed anxiety and/or insomnia stored images and sounds may be displayed in the patient's room to reproduce a home environment, reduce anxiety and promote sleep. Environmental supplement may include videos and night time sounds in the patient's home. Systems and methods to display stored images and sounds in a medical environment can be adapted for use with the system described herein (see e.g., U.S. Patent Application No. 2007/0176920, which is incorporated herein by reference).

As the patient's anxiety eases, her physiological readings, for example an EEG reading of an alpha or theta wave, electrooculogram readings from the video, and/or readings from the Actiwatch indicate she is beginning to get sleepy. The system's clock indicates an appropriate time for her to enter a sleep cycle based on input data from her health records regarding her chronotype to be morning type (see e.g., Adan et al., Chronobiology International, 29(9): 1153-1175, (2012) Circadian Typology: A Comprehensive Review). The system provides an audiovisual immersion supplement in which the room's lights dim and a sky (such as the sky from sensors in the nearby park or stored or generated images) is projected through the room. Over time the sky darkens, and stars are projected until her EEG measures theta waves indicating she has entered stage 3 sleep, at which time the immersive program is suspended. As the night progresses and her EEG and actigraph sensors detect signs of awakening at a time the system's clock determines to be appropriate, an immersive audiovisual display of a dawn sky is projected, slowly lightening and the sound of chirping birds slowly increases. Once fully awake, the patient can cancel the program manually.

Environmental supplement may also include fresh air input sourced directly from the external environment. Methods to control fresh air and measure oxygen, carbon dioxide, humidity and temperature can be adapted for use with the embodiments described herein (see e.g., U.S. Patent Application No. 2009/0065596, which is incorporated herein by reference). Systems and devices to control air: flow, temperature, humidity, oxygen and carbon dioxide levels are available from Honeywell, Golden Valley, Minn. Environmental supplement may include specific aromatherapy or other scents matching those of the home environment or favorite foods (e.g., cinnamon, vanilla, lilac, popcorn, baking bread, floral, fruity, ocean, etc.); they may be provided by a scent diffuser device (scents and scent diffusers are available from ScentAir Corp., Charlotte, N.C.). In response to sensor input, the system output may also include mechanical functions to promote sleep, ease pain or encourage activity and exercise.

The patient's bed may be motorized and controlled by the computer system. For example, if the computer-controlled system senses pain or discomfort in the patient (e.g., analysis of video taken of the patient or biological sensors in the bed or in the vicinity of the bed) then it may respond by moving the bed and changing the position/posture of the patient. Alternatively the system may vibrate the bed to encourage relaxation or to massage aching muscles. If the system senses the patient is drowsy (e.g., video input, EEG signals) or calculates sleep is required based on video monitoring, then it may respond by lowering the lights, reclining the bed, and playing restful sounds, e.g., ocean waves or rain falling.

### Prophetic Example 2

### Method and System to Promote Wellness for an Elderly Patient in a Hospital

An elderly patient is admitted to an acute care hospital with congestive heart failure. The patient is assigned to a room with a computer-controlled system to promote wellness during and after the patient's hospital stay. The system dynamically responds to inputs from electronic health records, caregivers, and physical, medical and environmental sensors with audio, visual and environmental supplement to create a healthy environment and low stress experience for the patient.

Upon admission to the hospital, data from the patient's medical record including the names of attending physicians, immediate family and friends are transferred to the system computer. Mobile phone and internet connections with the patient's family or friends, as well as personal products or personal habits are captured by the system as part of the patient's personal profile. The patient's past and future treatment plans including diagnostic tests, procedures and medications (with dose and schedule) are also entered into the computer. After admission to the hospital room the patient's physician reviews the intended treatment plan, diagnostic tests and medications as well as a tentative schedule for future visits by the physician and any other caregivers, e.g., doctors, nurses, technologists etc. The system computer captures the treatment plan, scheduled caregiver visits and programs alerts for the treatment plan events, as well as any additional personal profile data for matching with the Environmental Supplement database. For example, to treat heart failure the patient may be prescribed an angiotensin converting enzyme (ACE) inhibitor (e.g., Captopril™); a beta-blocker (e.g., Carvedilol™) and a diuretic (e.g., Bumetanide™)

To promote mental and emotional health the responsive dynamic system may create a virtual environment based on the patient's preferences or previous experience. Environmental supplement output derived from external sites is displayed in the patient's room. For example, if a tropical Hawaiian environment is preferred by the patient the system may use stored images, video, full spectrum lighting, controlled air flow and immersive audio visual systems to display a location or locations in Hawaii. The system may respond dynamically to external environmental sensors and change the environmental supplement as indicated by computer algorithm, by set program (e.g., at certain intervals) or as requested by the patient. For example environmental sensors near the hospital room may detect changes in daylight, precipitation, wind and temperature which are relayed to the system and used to inform programmed dynamic changes in the virtual Hawaiian environmental display. A dynamic response to external sensors at sundown may be an immersive audiovisual display of a Hawaiian sunset on the walls and ceiling of the patient's room and controlled lighting, temperature and ventilation to create a restful tropical environment. As the evening progresses into night, the audiovisual display is dynamically altered over time to display the night sky. Alternatively, distal external sensors, including video cameras, temperature sensors, and wind sensors in Hawaii may transmit real time audiovisual input to the responsive dynamic system for display in the patient's room in conjunction with lighting, ventilation and temperature input. Alternatively, data broadcast from public sensors over the internet regarding environmental parameters at a given time are captured by the system as environmental input and are used to inform the programmed dynamic changes.

To promote well-being and successful recovery upon release from the hospital the responsive system provides audiovisual and environmental cues for the patient. For example if the patient is not eating properly, as sensed by the video monitoring system, the system may display cooking shows with heart healthy foods that are available on the menu at the hospital and simultaneously provide aromas of favorite foods. If the patient is not moving about and needs to exercise, as sensed by the video monitors, the system may display exercises the patient can do in bed and/or after getting out of bed to promote ambulation. As the patient exercises, a motion-capture system (e.g., Kinect from Microsoft, Redmond, Wash.) captures the actions of the patient and the information is relayed to the system. The system provides an audiovisual immersion experience that incorporates the movements of the patient. For example if the patient is walking in place, the immersive environment can mimic the patient's home (e.g., using real time sensors) so that it appears the patient is walking through the home.

Likewise, if the video monitor system senses the patient is not exercising and moving enough then it may prompt the patient's caregivers to provide encouragement and if necessary, prodding. Also the responsive system can transmit patient requests for help in getting up or exercising by alerting caregivers through the interactive audio-video system. Prior to the patient leaving the hospital, the responsive system can review the post-hospital treatment plan including medication, nutrition, exercise, and future doctor visits. In addition, for support the treatment plan can be shared with the patient's family or friends via a bidirectional video communication system (see e.g., U.S. Pat. No. 8,520,050 Ibid. and U.S. Pat. No. 6,425,764 Ibid.). Moreover, the responsive dynamic system may begin training and education of the patient for departure from the hospital and healthy recovery as soon as treatment and rehabilitation plans are identified by the patient's physicians and caregivers.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1.-40. (canceled)

41. A system, comprising:
one or more computing devices having a non-transitory signal bearing medium operable to
accept data related to a subject in an altered Environmental Supplement location;
compare the data related to the subject to at least one Environmental Supplement dataset for enhancing the subject's healthcare setting;
select at least one Environmental Supplement that correlates as beneficial to the subject based on the comparison;
generate output related to the selected at least one Environmental Supplement.

42. The system of claim 41, further including providing instructions for altering the subject's surroundings to correspond to the selected Environmental Supplements if a condition threshold has been satisfied.

43. The system of claim 42, wherein the condition threshold includes one or more of time, change in the subject's status, or request by the subject.

44. The system of claim 42, wherein the output includes one or more suggestions or options for one or more Environmental Supplement determined to be beneficial to the subject's health.

45. The system of claim 41, wherein the one or more Environmental Supplements include one or more customized settings for at least one of temperature; lightness or darkness; humidity; vibration or other movement, influx of fresh air from outdoors or filtered air, images, sounds, or scents.

46. The system of claim 41, wherein the devices for providing one or more Environmental Supplements include at least one of an audio delivery device or visual display device.

47. The system of claim 46, wherein the audio delivery device includes a speaker or speaker system.

48. The system of claim 47, wherein the speaker or speaker system includes surround sound or pan sound.

49. The system of claim 41, wherein the system includes one or more devices for providing one or more Environmental Supplements including at least one of an audio delivery device or visual display device.

50. The system of claim 49, wherein the audio delivery device includes a speaker or speaker system.

51. The system of claim 50, wherein the speaker or speaker system includes surround sound or pan sound.

52. The system of claim 50, wherein the audio delivery device includes at least one of a sound dampening, white noise delivery, or noise-cancellation device.

53. The system of claim 50, wherein the visual display device includes at least one monitor, projector, immersive visualization display, dimmable machine information display, or projection screen.

54. The system of claim 53, wherein the visual display device includes at least one of a 2-dimensional, 3-dimensional, holographic, illumination, immersive, dynamic, stereoscopic, or surface changing technology.

55. The system of claim 53, wherein the visual display device includes at least one of rear projection screen, high resolution screen, gigapixel screen, LED display or LCD display.

56. The system of claim 53, wherein the visual display device includes at least one of modular, array, or tiled screen or display.

57. The system of claim 53, wherein the visual display device includes at least one of a retractable or movable screen or display.

58. The system of claim 53, wherein the projection screen is at least one of a forward projecting, rear projecting, illumination, dynamic projection, stereoscopic projection, surface changing projection, retractable screen, track-run screen, locatable screen, or stationary screen.

59. The system of claim 58, wherein the projection screen includes at least a portion of a wall, a ceiling, a floor, a furniture, or equipment a wall or furniture of the subject's setting.

60. The system of claim 58, wherein the projection screen includes at least one of a planar surface, soft surface, curved surface, or angular surface.

61. The system of claim 58, wherein the retractable screen is at least one of telescoping, accordion, or flap screen.

62. The system of claim 58, wherein the projection screen includes at least one of wheels, motors, mechanisms, transmitters, receivers, or circuitry.

63. The system of claim 41, wherein the data related to the subject includes at least one of personal health records, input from one or more sensors, input from at least one healthcare worker, or input from the subject.

64. The system of claim 63, wherein the one or more sensors include at least one of an EEG, EKG, pupillometer, blood pressure, ultrasound, bioimpedance, infrared thermometry, audio, visual, acoustic sensors, optical sensors, electromagnetic energy sensors, image sensors, photodiode arrays, charge-coupled devices (CCDs), complementary metal-oxide-semiconductor (CMOS) devices, optical recognition sensors, infrared sensors, radio frequency component sensors, thermo sensors, pressure sensors, or three-dimensional sensors.

65.-67. (canceled)

68. The system of claim 41, wherein the data related to the subject includes at least one of sensed data, stored data, or broadcast data.

69. The system of claim 41, wherein the selected Environmental Supplements are altered based on cyclic aspects correlated with the data related to the subject or a specific time period.

70. The system of claim 69, wherein the cyclic aspects include cycling one or more Environmental Supplements on a regular schedule as determined by the data related to the subject or a specific time period.

71. The system of claim 41, further including providing instructions for altering the subject's surroundings to correspond to the selected Environmental Supplements if a condition threshold has been satisfied.

72. The system of claim 71, wherein the condition threshold includes one or more of time, change in the subject's status, or request by the subject.

73. The system of claim 41, wherein the accepting data includes continuous input of data related to the subject.

74. The system of claim 41, wherein the Environmental Supplement includes mimicry of a dynamic spatial or temporal image.

75. The system of claim 74, wherein the dynamic spatial or temporal image includes at least one of an image of daylight turning to darkness, traveling on land, air, or sea.

76. The system of claim 41, wherein accepting the data related to the subject is conducted by remote control.

77. The system of claim 41, wherein accepting the data related to the subject is conducted by manual input.

78. The system of claim 41, wherein the setting of the subject includes at least one of a labor/delivery room, neonate room, intensive care unit, emergency room, psychiatric ward/room, pediatric ward/room, diagnostic ward/room, examination ward/room, cancer treatment center, Alzheimer's unit, eldercare unit, or long term care facility with a room, apartment, or other living area occupied by a subject.

79.-114. (canceled)

115. A method executed on a computing device, comprising:
accepting data related to a subject;
converting the data related to a subject into a personal profile;
comparing the subject's personal profile to at least one Environmental Supplement dataset for dynamically enhancing the subject's healthcare setting;
selecting at least one Environmental Supplement that correlates as beneficial to the subject based on the comparison;
generating output related to the selected at least one Environmental Supplement.

116. (canceled)

117. A method executed on a computing device, comprising:
accepting a first set of data related to a subject;
comparing accepted data related to the subject with an Environmental Supplement dataset;
selecting at least one Environmental Supplement determined to be beneficial to the subject's health based on the comparison;
generating an output related to the selected at least one Environmental Supplement;
transmitting one or more signals in response to the generated output.

118. The method of claim 117, further including:
recording the results of accepting the first set of data related to a subject;
repeating accepting a second set of data related to the same subject, and comparing the accepted second set of data with an Environmental Supplement dataset until a comparison satisfies a threshold condition;
generating an output related to the satisfaction of the threshold condition.
